(19) United States
(12) Patent Application Publication
(10) Pub. No.: US 2023/0390022 A1
(43) Pub. Date: Dec. 7, 2023

(54) NON-INVASIVE SEMI-PERMANENT SKIN MARKER

(71) Applicant: Radtat, LLC, Lenoir City, TN (US)

(72) Inventor: Zachary Allen Dutton, Lenoir City, TN (US)

(21) Appl. No.: 18/329,625

(22) Filed: Jun. 6, 2023

Related U.S. Application Data
(60) Provisional application No. 63/349,409, filed on Jun. 6, 2022.

(51) Int. Cl. A61B 90/00

(52) U.S. Cl. CPC A61B 90/39; A61B 2090/3933; A61B 2090/3987; A61B 2090/3991

(57) ABSTRACT

Various implementations include a method of applying a surgery reference marker to skin of a patient. The method includes providing a medical scanner having at least one positioning laser; disposing the patient in a desired position relative to the medical scanner such that the at least one positioning laser illuminates a portion of the skin of the patient; disposing a temporary tattoo on the portion of the skin of the patient, the temporary tattoo having at least one positioning indicator configured to be applied to the skin of the patient, wherein the temporary tattoo is disposed such that the at least one positioning indicator indicates a position of the at least one positioning laser; and applying the temporary tattoo such that the at least one positioning indicator is transferred to the skin of the patient.

### BACKGROUND

Some medical scanners require precise positioning of a portion of the body of a patient to produce a desired image. Furthermore, many procedures require a patient to return to the medical scanner after one or more days for subsequent imaging of the same portion of the body. In order for the two or more images produced from the medical scanner to be consistent with each other, the portion of the body to be scanned must be disposed in the same position and orientation relative to the medical scanner for each scan.

To accomplish this, many medical scanners include positioning laser sources that produce one or more laser beams that are configured to illuminate the portion of the body of the patient to be imaged. Thus, if the portion of the body of the patient is disposed in the same position and orientation such that these laser beams illuminate the same locations on the skin of the patient, then the medical scanner will image the same portion of the body each scan.

To ensure that the laser beams illuminate the same locations on the skin of the body, users have utilized skin markings such as marker ink, adhesive stickers, and other temporary indicators. However, these indicators are too temporary and require excess care to ensure that they are not removed between scans.

For longer treatments requiring many scans over an extended period of time, some patients opt for tattoos for indicators. However, the tattoo indicators are permanent and will remain on the patient's skin long after the treatment is completed.

### SUMMARY

Various implementations include a method of applying a surgery reference marker to skin of a patient. The method includes providing a medical scanner having at least one positioning laser; disposing the patient in a desired position relative to the medical scanner such that the at least one positioning laser illuminates a portion of the skin of the patient; disposing a temporary tattoo on the portion of the skin of the patient, the temporary tattoo having at least one positioning indicator configured to be applied to the skin of the patient, wherein the temporary tattoo is disposed such that the at least one positioning indicator indicates a position of the at least one positioning laser; and applying the temporary tattoo such that the at least one positioning indicator is transferred to the skin of the patient.

In some implementations, the medical scanner includes a computerized tomography (CT) scanner. In some implementations, disposing the patient in a desired position relative to the medical scanner includes positioning the patient on a table associated with the medical scanner.

In some implementations, the at least one positioning laser produces a beam along a central axis. In some implementations, the beam has a crosshair shape in a cross-section perpendicular to the beam axis. In some implementations, the at least one positioning laser includes a three-point laser setup.

In some implementations, the at least one positioning indicator includes a crosshair.

In some implementations, the temporary tattoo includes a gardenia-based ink. In some implementations, the at least one positioning indicator includes an ink that is configured to bind to protein of the skin of the patient over time.

In some implementations, the temporary tattoo includes a tattoo portion including the at least one positioning indicator and an adhesive portion. In some implementations, the adhesive portion is coupled to the tattoo portion such that the adhesive portion overhangs an edge of the tattoo portion and does not cover the at least one positioning indicator. In some implementations, the adhesive portion is coupled to opposing edges of the tattoo portion. In some implementations, the adhesive portion fully extends along all edges of the tattoo portion.

In some implementations, the method further includes applying the adhesive portion to the skin of the patient after disposing the temporary tattoo on the portion of the skin of the patient.

In some implementations, applying the temporary tattoo such that the at least one positioning indicator is transferred to the skin of the patient further includes wetting the temporary tattoo.

Various other implementations include a temporary tattoo. The temporary tattoo includes a tattoo portion and an adhesive portion. The tattoo portion has at least one positioning indicator configured to be applied to skin. The adhesive portion is coupled to the tattoo portion such that the adhesive portion overhangs an edge of the tattoo portion and does not cover the at least one positioning indicator.

In some implementations, the adhesive portion is coupled to opposing edges of the tattoo portion. In some implementations, the adhesive portion fully extends along all edges of the tattoo portion.

In some implementations, the at least one positioning indicator includes a crosshair.

In some implementations, the temporary tattoo includes a gardenia-based ink. In some implementations, the at least one positioning indicator includes an ink that is configured to bind to protein of the skin of the patient over time.

### BRIEF DESCRIPTION OF DRAWINGS

Example features and implementations are disclosed in the accompanying drawings. However, the present disclosure is not limited to the precise arrangements and instrumentalities shown.

FIG. 1A is a front view of a temporary tattoo, according to one implementation.

FIG. 1B is a side view of the temporary tattoo of FIG. 1A.

FIG. 2 is a perspective view of three implementations of temporary tattoos.

FIG. 3 is a perspective view of two implementations of temporary tattoos.

FIG. 4A is a front view of a temporary tattoo, according to another implementation.

FIG. 4B is a side view of the temporary tattoo of FIG. 4A.

FIG. 4C is a rear view of the temporary tattoo of FIG. 4A.

FIG. 5 is a perspective view of a temporary tattoo, such as the one shown in FIG. 1A, being applied to the skin of a patient.

### DETAILED DESCRIPTION

The devices, systems, and methods disclosed herein provide for a non-invasive, semi-permanent or temporary tattoo marking used for patient position in radiologic sciences, radiation therapy, oncology, and other medical fields. The devices, systems, and methods disclosed herein are designed to locate simulation setup marks, treatment marks, skin marks, patient marks, reference marks, as well as daily treatment and patient positioning setups. The devices disclosed herein are designed to be applied to the skin and last for at least two weeks.

Various implementations include a method of applying a surgery reference marker to skin of a patient. The method includes providing a medical scanner having at least one positioning laser; disposing the patient in a desired position relative to the medical scanner such that the at least one positioning laser illuminates a portion of the skin of the patient; disposing a temporary tattoo on the portion of the skin of the patient, the temporary tattoo having at least one positioning indicator configured to be applied to the skin of the patient, wherein the temporary tattoo is disposed such that the at least one positioning indicator indicates a position of the at least one positioning laser; and applying the temporary tattoo such that the at least one positioning indicator is transferred to the skin of the patient.

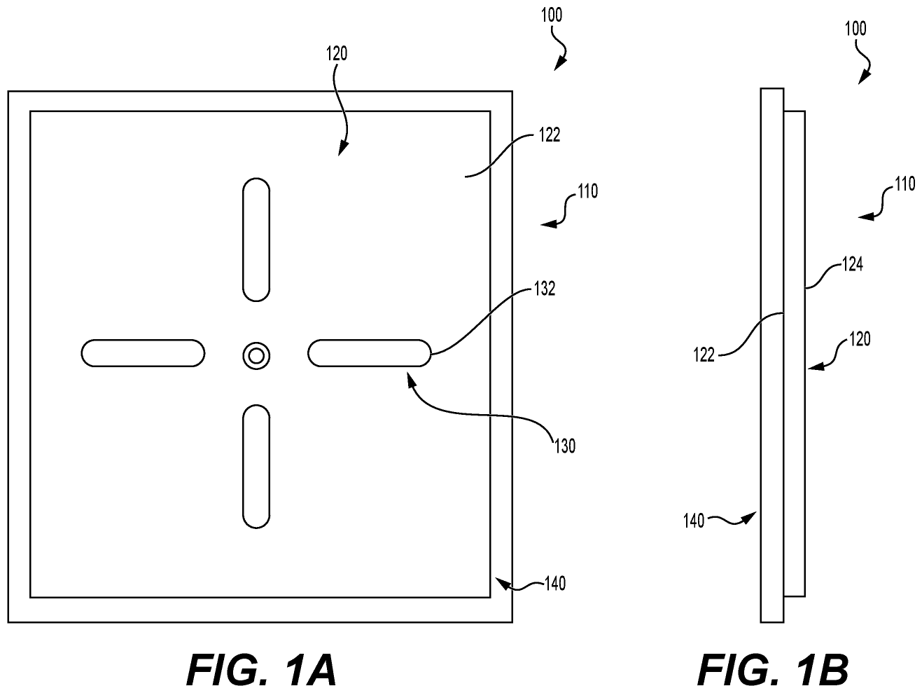

FIGS. 1A and 1B show a temporary tattoo 100 including aspects according to various implementations. The temporary tattoo 100 includes a tattoo portion 110 having a backing portion 120 and a cover portion 140. The backing portion 120 is made of a water-absorbent material, such as transfer paper or any wood pulp product. The backing portion 120 has a first backing side 122 and a second backing side 124 opposite the first backing side 122. The first backing side 122 of the backing portion 120 includes at least one positioning indicator 130 that includes a temporary tattoo ink 132. The ink 132 shown in FIGS. 1A and 1B is a gardenia-based ink. When applied to the skin of a patient, the gardenia-based ink 132 binds to the protein of the skin of the patient over time, as discussed below. However, in some implementations, the positioning indicator includes any type of ink or other marking material that semi-permanently marks the skin of the patient.

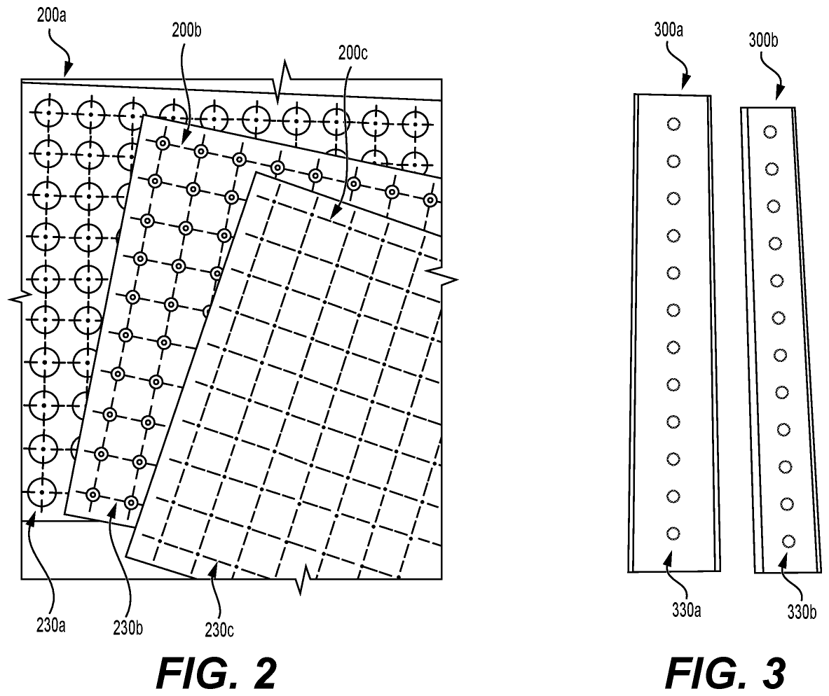

The positioning indicator 130 of the backing portion 120 can include any design or shape indicator. For example, FIG. 2 includes three temporary tattoos 200a, 200b, 200c with different positioning indicators 230a, 230b, 230c that form a crosshair pattern: a first crosshair pattern with a large concentric circle, a second crosshair pattern with a small concentric circle, and a third crosshair pattern without a concentric circle. FIG. 3 shows two other temporary tattoos 300a, 300b with different positioning indicators 330a, 330b that form a dot pattern: a first dot pattern with a large dot and a second dot pattern with a small dot. In some implementations, the temporary tattoo includes any other positioning indicator patterns, such as straight lines, curved lines, any outlined or solid shapes, or any combination thereof.

The cover portion 140 of the temporary tattoo 100 shown in FIGS. 1A and 1B is a thin plastic film that is coupled to the first backing side 122 of the backing portion 120 and the at least one positioning indicator 130. The cover portion 140 is designed to protect the positioning indicator 130 of the backing portion 120 to prevent the positioning indicator 130 from being damaged prior to application. The cover portion 140 can be removed from the first backing side 122 of the backing portion 120 to expose the positioning indicator 130.

The cover portion 140 shown in FIGS. 1A and 1B are clear or transparent such that the user can see the positioning indicator 130 on the backing portion 120 through the cover portion 140. This allows a user to see where the positioning indicator 130 is located before removing the cover portion 140. However, in some implementations, the cover portion is translucent or opaque.

As shown in FIGS. 2 and 3, two or more temporary tattoos 200a, 200b, 200c, 300a, 300b may be included in a single sheet of temporary tattoos. In this configuration, individual temporary tattoos 200a, 200b, 200c, 300a, 300b can be cut from the sheet such that each individual temporary tattoo 200a, 200b, 200c, 300a, 300b includes one or more positioning indicators 230a, 230b, 230c, 330a, 330b.

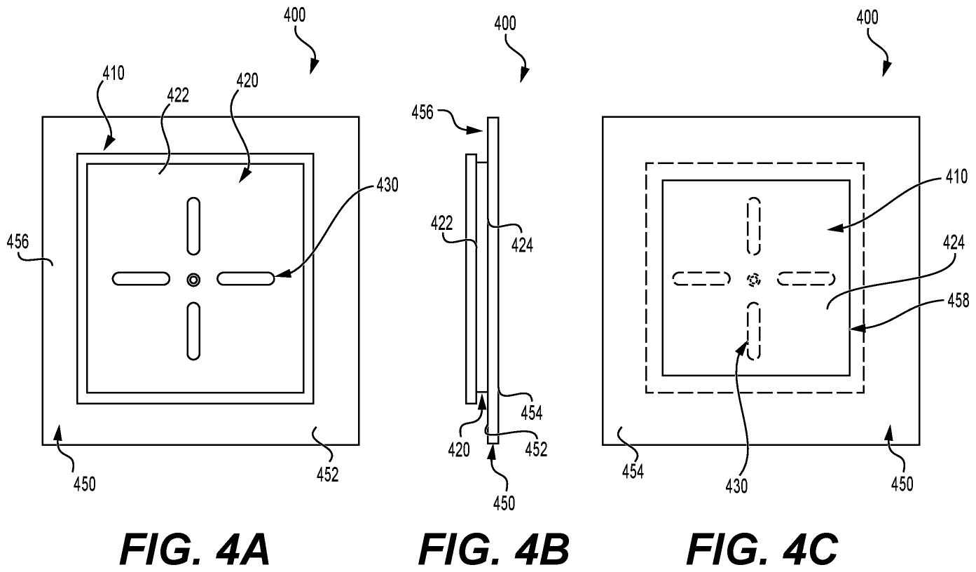

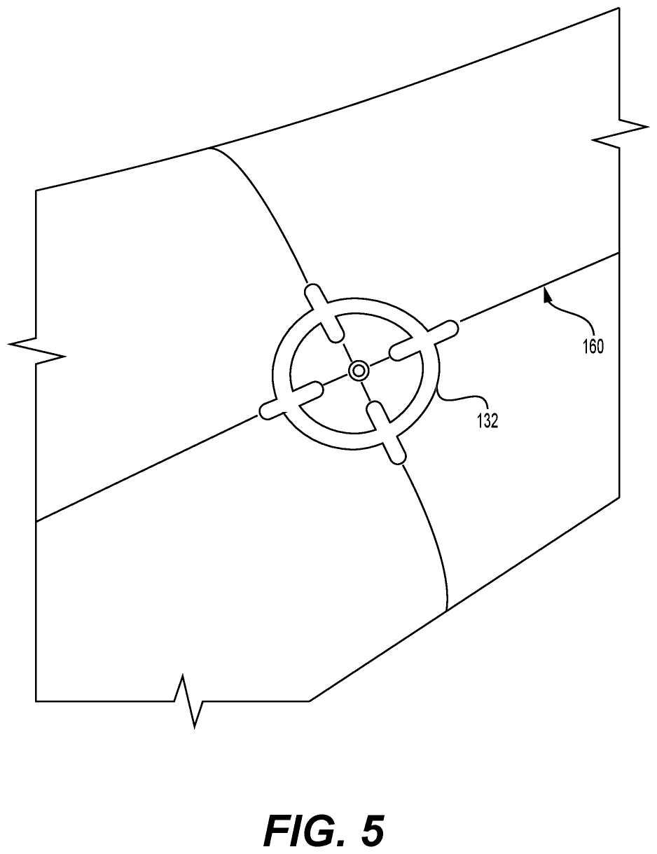

FIGS. 4A-4C shows another temporary tattoo 400 including aspects according to various implementations. The temporary tattoo 400 includes many features that are similar to features of the temporary tattoo 100 shown in FIGS. 1A and 1B. Thus, similar reference numbers are used for the temporary tattoo 400 shown in FIGS. 4A-4C to indicate similar features to those of the temporary tattoo 100 shown in FIGS. 1A and 1B.

The temporary tattoo 400 shown in FIGS. 4A-4C includes a tattoo portion 410, as described above, and an adhesive portion 450.

The adhesive portion 450 shown in FIGS. 3A-3C has a first adhesive side 452 and a second adhesive side 454 opposite the first adhesive side 452. The first adhesive side 452 includes an adhesive 456 disposed along at least a portion of the first adhesive side 452. The first adhesive side 452 defines an opening 458 extending to the second adhesive side 454.

The first adhesive side 452 is coupled to the second backing side 424 of the backing portion 420 such that a projection of the positioning indicator 430 from the first backing side 422 onto the second backing side 424 is fully or partially disposed within the opening 458 of the adhesive portion 450. Also, the backing portion 420 is sized such that at least a portion of the first adhesive side 452 overhangs an edge of the backing portion 420.

Although the adhesive portion 450 shown in FIGS. 4A-4C defines an opening 458 and fully extends along all edges of the tattoo portion 410, in some implementations, the adhesive portion may not define an opening. In some implementations, the adhesive portion only overhangs a single edge of the backing portion. In some implementations, the adhesive portion overhangs two edges of the backing portion, such as opposing edges of the backing portion.

In some implementations, the device includes two or more adhesive portions, and each adhesive portion extends from, or overhangs, one or more edges of the backing portion.

In some implementations, the adhesive portion is coupled directly onto the edge of the backing portion such that the adhesive portion extends from the edge of the backing portion.

In some implementations, the device does not include an adhesive portion and only includes the tattoo portion. In some implementations, an adhesive is disposed directly onto the backing portion. In some implementations, the device does not include adhesive.

In some implementations, the adhesive portion is coupled to the first backing side. In such implementations, the first adhesive side may define an opening extending to the second adhesive side, and the second adhesive side is coupled to the first backing side such that the positioning indicator is fully or partially disposed within the opening of the adhesive portion. In some implementations, the adhesive portion may not need to overhang the edges of the backing portion since the adhesive on the first adhesive side is fully exposed.

In some implementations, the cover portion of the tattoo portion covers the first backing side of the backing portion and the adhesive of the first adhesive side or the adhesive portion.

In use, the patient is placed in a desired position relative to a medical scanner having at least one positioning laser. For example, in some implementations, the medical scanner includes a CT scanner. In some implementations, disposing the patient in a desired position relative to the medical scanner includes positioning the patient on a table associated with the medical scanner.

The patient is positioned relative to the medical scanner such that at least one positioning laser of the medical scanner produces a laser beam 160 that illuminates a portion of the skin of the patient. An example beam 160 of a positioning laser is shown in FIG. 4.

In some implementations, the at least one positioning laser produces a beam 160 along a central axis. In some implementations, the beam has a crosshair shape in a cross-section perpendicular to the beam axis. In some implementations, the at least one positioning laser includes a three-point laser setup.

The portion of skin illuminated by the positioning laser beam 160 should be cleaned and thoroughly dried in preparation for the application of the temporary tattoo 100.

If the temporary tattoo 100 is included in a sheet of temporary tattoos, the individual temporary tattoo 100 is cut from the sheet such that one or more positioning indicators 130 are included on the temporary tattoo 100. The cover portion 140 is then peeled or otherwise removed from the first backing side 122 of the backing portion 120 to expose the positioning indicator 130.

The first backing side 122 of the temporary tattoo 100 is then disposed on the portion of the skin of the patient such that the positioning indicator 130 of the temporary tattoo 100 is disposed against the skin of the patient in a desired position relative to the illumination from the positioning laser beam 160. Thus, the temporary tattoo 100 is disposed such that the at least one positioning indicator 130 indicates a position of the at least one positioning laser beam 160.

For implementations in which the temporary tattoo 400 includes an adhesive portion 450, the first adhesive side 452 is facing the skin of the user such that the adhesive 456 on the first adhesive side 452 abuts the skin. The user can apply pressure along the second adhesive side 454 of the adhesive portion 450 to cause the adhesive 456 to couple to the skin of the user.

A wet towel or other absorbent article is applied to the second backing side 124 of the backing portion 120 of the temporary tattoo 100. The wet towel or other absorbent article is held against the second backing 124 side for 10 seconds to 15 seconds or until enough moisture from the wet towel or other absorbent article is absorbed into the backing portion 120 to transfer the ink 132 of the at least one positioning indicator 130 onto the skin of the patient. The wet towel or other absorbent article is then removed from the second backing side 124 of the backing portion 120 of the temporary tattoo 100.

In some implementations of the method, the backing portion can be immediately removed after the ink of the at least one positioning indicator is transferred onto the skin of the patient. The skin can then be dried, or allowed to dry, of any excess moisture, as shown in FIG. 4.

In some implementations of the method, tape, a bandage, or any other type of adhesive or means of coupling is applied to the backing portion to couple the backing portion to the skin of the patient. The coupling of the backing portion to the skin of the patient allows the ink of the at least one positioning indicator to fully transfer to the skin of the patient over time while covering and protecting the ink from being disturbed.

In some implementations of the method in which the temporary tattoo includes an adhesive portion, the adhesive on the first adhesive side of the adhesive portion couples the backing portion to the skin of the patient. Similar to above, the coupling of the backing portion to the skin of the patient allows the ink of the at least one positioning indicator to fully transfer to the skin of the patient over time while covering and protecting the ink from being disturbed.

In some cases, it is desirable to apply two or more temporary tattoos to indicate the position of the at least one positioning laser. If two or more temporary tattoos are desired, the above method can be repeated.

Once the ink of the at least one positioning indicator is transferred to the skin of the patient, the ink will be light in color. However, 24 to 48 hours after the ink has been applied, the ink will bind with the protein of the skin of the patient and begin to darken.

Because the ink of the at least one positioning indicator binds to the protein of the skin of the patient, the ink will not be removed from the skin with the application of soap and water. Typically, the ink of the at least one positioning indicator can last up to 2-3 weeks with proper care. However, the ink of the at least one positioning indicator can be removed with isopropyl alcohol (i.e., rubbing alcohol) or mineral oil (i.e., baby oil).

It is understood that the at least one positioning indicator of the temporary tattoos disclosed herein can include any shapes, colors, sizes, dimensions, prints, designs, numbers, characters, patterns, or any other features that are used as skin markers for patient positioning or localization in healthcare. The at least one positioning indicators of the temporary tattoos disclosed herein can include any ink types, colors, temporary or semi-permanent durations, or application types.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A method of applying a surgery reference marker to skin of a patient, the method comprising:
providing a medical scanner having at least one positioning laser;
disposing the patient in a desired position relative to the medical scanner such that the at least one positioning laser illuminates a portion of the skin of the patient;
disposing a temporary tattoo on the portion of the skin of the patient, the temporary tattoo having at least one positioning indicator configured to be applied to the skin of the patient, wherein the temporary tattoo is disposed such that the at least one positioning indicator indicates a position of the at least one positioning laser; and
applying the temporary tattoo such that the at least one positioning indicator is transferred to the skin of the patient.

2. The method of claim 1, wherein the medical scanner comprises a computerized tomography (CT) scanner.

3. The method of claim 1, wherein disposing the patient in a desired position relative to the medical scanner comprises positioning the patient on a table associated with the medical scanner.

4. The method of claim 1, wherein the at least one positioning laser produces a beam along a central axis, the beam having a crosshair shape in a cross-section perpendicular to the beam axis.

5. The method of claim 1, wherein the at least one positioning laser comprises a three-point laser setup.

6. The method of claim 1, wherein the at least one positioning indicator comprises a crosshair.

7. The method of claim 1, wherein the temporary tattoo comprises a gardenia-based ink.

8. The method of claim 1, wherein the at least one positioning indicator comprises an ink that is configured to bind to protein of the skin of the patient over time.

9. The method of claim 1, wherein the temporary tattoo comprises a tattoo portion including the at least one positioning indicator and an adhesive portion, wherein the adhesive portion is coupled to the tattoo portion such that the adhesive portion overhangs an edge of the tattoo portion and does not cover the at least one positioning indicator.

10. The method of claim 9, wherein the adhesive portion is coupled to opposing edges of the tattoo portion.

11. The method of claim 10, wherein the adhesive portion fully extends along all edges of the tattoo portion.

12. The method of claim 9, further comprising applying the adhesive portion to the skin of the patient after disposing the temporary tattoo on the portion of the skin of the patient.

13. The method of claim 1, wherein applying the temporary tattoo such that the at least one positioning indicator is transferred to the skin of the patient further comprises wetting the temporary tattoo.

14. A temporary tattoo comprising:
a tattoo portion having at least one positioning indicator configured to be applied to skin; and
an adhesive portion coupled to the tattoo portion such that the adhesive portion overhangs an edge of the tattoo portion and does not cover the at least one positioning indicator.

15. The temporary tattoo of claim 14, wherein the adhesive portion is coupled to opposing edges of the tattoo portion.

16. The temporary tattoo of claim 15, wherein the adhesive portion fully extends along all edges of the tattoo portion.

17. The temporary tattoo of claim 14, wherein the at least one positioning indicator comprises a crosshair.

18. The temporary tattoo of claim 14, wherein the temporary tattoo comprises a gardenia-based ink.

19. The temporary tattoo of claim 14, wherein the at least one positioning indicator comprises an ink that is configured to bind to protein of the skin of the patient over time.
